(19) United States
(12) Patent Application Publication
(10) Pub. No.: US 2010/0076537 A1
(43) Pub. Date: Mar. 25, 2010

(54) ELECTRODE CONFIGURATIONS FOR ELECTROCHEMICALLY ACTIVATED SYSTEMS

(76) Inventors: Edwin Jager, Linkoeping (SE); Magnus Krogh, Linkoeping (SE); Daniel Carlsson, Stockholm (SE); Olle Inganaes, Linkoeping (SE)

Correspondence Address: SCHIFF HARDIN, LLP;PATENT DEPARTMENT, 233 S. Wacker Drive-Suite 6600, CHICAGO, IL, 60606-6473, US

(21) Appl. No.: 12/516,945

(22) PCT Filed: Mar. 20, 2007
(86) PCT No.: PCT/EP07/02536

§ 371 Date: May 29, 2009

Related U.S. Application Data
(60) Provisional application No. 60/787,162, filed on Mar. 30, 2006.

(30) Foreign Application Priority Data
Mar. 30, 2006 (SE) 0600734-8

(51) Int. Cl. A61N 1/05; C25D 17/10; C25B 11/04; H01R 43/00

(52) U.S. Cl. 607/116; 204/225; 204/291; 29/825

(57) ABSTRACT

An electrode for use in a liquid electrolyte is at least partially provided with a covering, the covering being ion permeable and electrically insulating. Systems and methods for preventing short circuiting of the electrodes are also disclosed. Also disclosed is a counter electrode for use with a working electrode comprising conducting polymer, in the presence of an electrolyte. The counter electrode comprises conducting polymer. Systems and methods for preventing ion depletion of the electrolyte are also disclosed.

### CROSS-REFERENCE TO RELATED APPLICATION

This PCT application claims the benefit of U.S. Provisional Patent Application Ser. No. 60/787,162, filed Mar. 30, 2006, which is incorporated herein by reference.

### BACKGROUND OF THE INVENTION

1. Field of the Invention

The present disclosure relates to electrode configurations for electrochemically activated systems. In particular, the disclosure relates to electrodes in such electrochemically activated systems, having coverings or coatings, to systems of such electrodes and to methods for operating such systems.

2. Description of the Related Art

During surgical procedures, where sensors and/or electrically actuated tools are used, high performance and well defined behavior are critical. Short circuits between electrodes and/or instruments, undesired reactions at electrode surfaces and the like are events which could lead to malfunction of the tool and severe damage to or even death of the patient.

Examples of such systems are surgical tools based on (electrical or) electrochemical principles such as disclosed in PCT Published International application no. WO 00/78222.

FIG. 1 schematically illustrates an electrochemical system 10. The system comprises a control unit 11 (for example, a potentiostat) an electrochemical cell 12 containing an electrolyte 13, a working electrode 14, counter electrode 15, and a reference electrode 16. The electrodes 14 through 16 may be connected to the control unit 11 by cables or wirelessly. The working electrode is typically the electroactive polymer actuator that may be an active part of a surgical tool.

An example of such a tool is provided in FIG. 2 (prior art, as shown in PCT published application no. WO 00/78222). The surgical tool 20 comprises a catheter or cannula 21 and a pair of tubular tweezers 22 that are inserted through the cannula/catheter 21 to reach the specific area for the operation to be performed. The tweezers 22 may be electrically actuated by electroactive polymer actuators.

Yet another example of such a tool is shown in FIG. 3. FIG. 3 schematically illustrates an elongated medical device 30 for insertion into the body, having a proximal end 31 and a distal end 32. The medical device 30 may be a lead, a catheter, or a guide wire. The medical device may comprise a non-controllable part 34, an electrically controllable portion 33, which functions as the working electrode, and a counter electrode 15a, 15b, 15c.

FIG. 3 also illustrates possible examples of where the counter electrode 15a, 15b, 15c may be placed in, near or on the medical device 30. Using a separate device 35 such as a second catheter, a guidewire, a lead and the like, the counter electrode 15c may be positioned near the first medical device 30. Alternatively, or as a complement, the counter electrode 15a may be placed on the non-controllable portion 34 of the device 30. Alternatively, or as a complement, the counter electrode 15b may be placed on an electrically insulated part of the controllable portion 33 of the device 30.

When using electrodes, which are to some extent movable relative to each other, there may be a risk of the electrodes coming into electrical contact with each other, thereby causing a short circuit. This is a problem known to those skilled in the art (see for instance Published US Patent Application No. US2005/0165439).

Medical devices of the above described type are often operated in small spaces or via a lumen extending into the body. Therefore, miniaturization of the tools is one important aspect of the development of this type of surgical tools. Both the small spaces where the tools operate, and the small size of the tool per se, can lead to constrictions on the functionality. The inventors have discovered that one such constriction is the amount of electrolyte, and therewith ions, that is available for the EAP (electro-active polymer) based tool when operating in such small spaces. Lack of ions may lead to reduced functionality, for example in terms of speed of actuation and actuation range. Also, operating in small areas/devices increases the aforementioned risk of short circuiting.

There is thus a need for even safer and more reliable electrochemically activated surgical tools.

For general details on electrochemistry reference is made to textbooks such as “Electrochemical Methods. Fundamentals and Applications”, A. J. Bard and L. R. Faulkner, ISBN 0-471-04372-9. An overview on electroactive polymers can be found in “Electroactive Polymers (EAP) Actuators as Artificial Muscles—Reality, Potential, and Challenges” 2nd ed. Y. Bar-Cohen (ed.) ISBN 0-8194-5297-1. More specific details on conducting polymer based (micro-) actuators can be found in Q. Pei and O. Inganäs, “Conjugated polymers and the bending cantilever method: electrical muscles and smart devices”, Advanced materials, 1992, 4(4), p. 277-278. and Jager et al., “Microfabricating Conjugated Polymer Actuators”, Science 2000 290: 1540-1545).

One class of EAPs (electro-active polymers) are conducting polymers. These are polymers with a backbone of alternating single and double bonds. These materials are semiconductors and their conductivity can be altered from insulating to conducting with conductivities approaching those of metals. Polypyrrole (PPy) is one such conducting polymer and will be taken here as an example.

PPy can be electrochemically synthesized from a solution of pyrrole monomers and a salt as is know to those skilled in the art. After synthesis PPy is in its oxidized, or also called doped, state. The polymer is doped with an anion A−.

PPy can be electrochemically oxidized and reduced by applying the appropriate potential to the material. This oxidation and reduction is accompanied with the transport of ions and solvents into and out of the conducting polymer. This redox reaction changes the properties of polypyrrole, such as the conductivity, color, modulus of elasticity and volume.

Two different schemes of redox are possible:

If PPy is doped with a large, immobile anion A− scheme 1 occurs, which schematically can be written as:

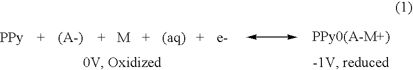

When PPy is reduced to its neutral state, cations M+ including their hydration shell and solvent are inserted into the material and the material swells. When PPy is oxidized again the opposite reaction occurs, M+ cations (including hydration shell and solvent) leave the material and its volume decreases.

If on the other hand PPy is doped with small, mobile anions a−, scheme 2 occurs:

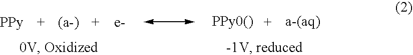

In this case the opposite behavior of scheme 1 occurs. In the reduced state, the anions leave the material and it shrinks. The oxidized state is now the expanded state and the reduced state the contracted. Non limiting example of ions A− is dodecylbenzene sulfonate (DBS-), of a− perchlorate (ClO4-), and of M+sodium (Na+) or lithium (Li+).

The ions (and solvent) that are transferred into and out of the conducting polymer are exchanged from an ion source/sink, i.e. the electrolyte 13. For reliable operation it is important that there is a good availability of ions in the electrolyte so that the functionality of the device is not limited by the ion concentration in the surrounding electrolyte.

### SUMMARY OF THE INVENTION

The present invention provides even safer and more reliable electrochemically activated surgical tools. In one embodiment, the present invention reduces the risk of short circuiting of electrochemically activated surgical tools, without compromising their function. In another embodiment, the present invention overcomes the problems associated with use of electrochemically activated tools in small spaces or low concentration electrolytes.

These advantages are wholly or partially met by devices, systems, and methods as described herein and as defined by the appended claims.

According to a first aspect, there is provided an electrode system for use in a liquid electrolyte, comprising a first substrate member, a second substrate member, a working electrode, arranged on one of the first and second substrate members, and a counter electrode, arranged on one of the first and second substrate members. The first and second substrate members are movable relative each other. In the alternative, or as a complement, the working electrode is movable relative to the counter electrode. At least one of the electrodes is at least partially provided with a covering, the covering being ion permeable and electrically insulating.

For purposes of this invention, “ion permeable” means sufficiently permeable for the electrode to perform its function. “Electrically insulating” means sufficiently insulating to prevent a short circuit if the electrode is contacted by another, e.g. metallic electrode or conducting polymer electrode.

The covering may be provided on an active part of the electrode. The covering may be in the form of a material layer, a coating, a wrapping, or a housing. Covering one of the electrodes, preferably the counter or auxiliary electrode with an ion conducting layer or ion exchange layer separates the ion conduction from the electrical conduction, thus preventing short circuits. By such an arrangement, a mechanical contact between the electrodes is prevented from leading to electrical contact between the electrode, whereby short circuiting of the electrodes is prevented or counteracted.

The covering may comprise a material, which is ion conducting, but electrically insulating. Alternatively, or as a complement, the covering may be provided as a coating on the electrode. Alternatively, or as a complement, the covering may be provided as a pre-formed part, which is attached to the electrode. Alternatively, or as a complement, the covering may be formed directly on the electrode. Alternatively, or as a complement, the covering may comprise pores or channels for transporting ions (and solvent). Hence, the material may be impermeable, but having pores or through channels sufficient to allow transport of ions and electrolyte. Alternatively, or as a complement, the covering may comprise a web of fibers or wires. Such a web may be a woven or a non-woven web, a mesh, etc.

The covering may be removably arranged on the electrode. There may be a gap between said electrode and the covering. The working electrode may be arranged on the first substrate member. The counter electrode may be arranged on the second substrate member. The working electrode and counter electrode may be arranged on said first substrate member, and said second substrate member may be electrically conducting. The first and second substrate members may form separate units.

The covering may be arranged on the working electrode. In the alternative, or as a complement, the covering may be arranged on the counter electrode. The electrode system may further comprise a reference electrode. Alternatively, or as a complement, the reference electrode, if any, may be provided with the covering.

According to a third aspect, there is provided a method for preventing short circuiting of a working electrode, arranged on one of a first and a second part, a counter electrode, arranged on one of said first and second parts, and optionally a reference electrode, arranged on one of said first and second parts, operating with an electrolyte, wherein the first and second parts are movable relative each other, and/or wherein the working electrode, the counter electrode or the reference electrode, if any, are movable relative to each other, the method comprising at least partially providing at least one of said working electrode, said counter electrode and said reference electrode with a covering that is ion permeable and electrically insulating.

According to a fourth aspect, there is provided a counter electrode for use with a working electrode comprising conducting polymer, in the presence of an electrolyte. The counter electrode comprises a conducting polymer.

Use of such a counter electrode may counteract depletion of ions in the electrolyte, and thereby counteract effects such as the need for over potentials for the redox process and long diffusion paths for the ions, leading to long response times and even decreased final expansion.

Coating the counter electrode with a conducting polymer may avoid undesired reactions, such as gas formation, at the electrode surface. Also, conducting polymer coatings can have favorable effects on performance, e.g. act as an ion source or lower the necessary potential span. It has been noticed by the inventors that metal ions e.g. Au ions are dissolved from the counter electrode and transferred to and deposited on top of the working electrode to form a thin metal layer. This deteriorated the functionality of the working electrode. Yet another advantage of covering the counter electrode with a conducting polymer is that this metal ion transfer and dissolution is prevented.

The conducting polymer of the counter electrode may be same as that of the working electrode. Alternatively, or as a complement, the conducting polymer of the counter electrode may be operable with a different scheme than that of the working electrode. Alternatively, or as a complement, the conducting polymer of the counter electrode may be of a different conducting polymer than that of the working electrode.

The conducting polymer may be provided as a coating on the counter electrode, covering all or part thereof. The counter electrode may be at least partially provided with an ion permeable and electrically insulating covering, such as the ones described with reference to the first three aspects above. The covering may at least partially cover the conducting polymer.

According to a fifth aspect, there is provided system comprising a working electrode, a counter electrode and an electrolyte, wherein the working electrode comprises a conducting polymer. The counter electrode also comprises a conducting polymer.

The conducting polymer of the counter electrode may be same as that of the working electrode. Alternatively, or as a complement, the conducting polymer of the counter electrode is operable with a different scheme than that of the working electrode. Alternatively, or as a complement, the conducting polymer of the counter electrode may be of a different conducting polymer than that of the working electrode.

The conducting polymer may be provided as a coating on the counter electrode, covering all or part thereof. The counter electrode may be at least partially provided with an ion permeable and electrically insulating covering, such as the ones described with reference to the first three aspects above. The covering may at least partially cover the conducting polymer.

A volume or ion concentration of the electrolyte may be so small that ion depletion in a vicinity of the working electrode of said electrolyte significantly reduces system functionality.

The volume of electrolyte may be less than the volume of conducting polymer on the counter electrode, multiplied by a ratio of the ion concentration of the conducting polymer to the ion concentration of the electrolyte, multiplied by a factor A, wherein the factor A is 20, preferably 10, 5, 2 or 1.

The electrolyte may be confined in a three dimensional space, at least one dimension of which being small enough to effectively limit an amount of ions available to for interaction with the working electrode, such that ion depletion of said electrolyte in a vicinity of the working electrode significantly reduces system functionality.

By “reduced system functionality” is meant, for example, a decrease in expansion rate or total expansion, or a current decrease, as compared to a system having a sufficient volume of electrolyte and/or amount of ions available in the electrolyte in the vicinity of the working electrode.

Also, in this context, the “vicinity” means the area or space surrounding the working electrode, and from which ions may be drawn in an amount and rate sufficient to provide an acceptable system functionality—the “acceptable” standard being dependent on the specific application.

The at least one dimension may be less than 10 times a thickness of the conducting polymer at the working electrode, or less than 5 times, or less than 2 times or even less than the thickness of the conducting polymer on the working electrode.

In the above described systems, the working electrode, the counter electrode and the reference electrode, if any, may be so closely spaced apart that they risk contacting each other during normal operation of the system.

The working electrode, the counter electrode, and the reference electrode, if any, may be provided on respective substrate members, which may be separate members.

The working electrode may, upon actuation, be expandable towards the counter electrode. The working electrode may form part of a bender, or bending element, which, upon actuation, bends towards the counter electrode.

In the above described systems, the substrate members may be substantially stationary relative to each other. Alternately, in the above described systems, the substrate members may be movable relative to each other. Further, in the above described systems, the substrate members may interact in a telescoping sliding or inter-sliding manner. In the above described systems, the working electrode, the counter electrode, and reference electrode, if any, may be provided in or on a tubular member. In the above described systems, the tubular member is adapted for insertion into a body lumen.

According to a sixth aspect, there is provided a method for preventing ion depletion in an electrochemical cell comprising a working electrode, a counter electrode and an electrolyte, wherein the working electrode comprises a conducting polymer. The method comprises providing a counter electrode comprising a conducting polymer.

Embodiments will now be described with reference to the appended drawings.

### BRIEF DESCRIPTION OF THE DRAWINGS

FIG. 1 is a block diagram that schematically illustrates an electrochemical system.

FIG. 2 is an enlarged cross section that schematically illustrates a prior art surgical tool based on electroactive polymer actuators.

FIG. 3 is a block diagram that schematically illustrates a prior art medical device based on electroactive polymer actuators.

FIGS. 4a-4b are side cross sectional views that schematically illustrate the first aspect according to the principles of the present invention.

FIGS. 5a-5c are side cross sectional view that schematically illustrate different embodiments of the covering of the first aspect of the present invention.

FIG. 6 is a graph that illustrates a cyclic voltammogram related to the first aspect.

FIGS. 7a-7d are side cross sectional views that schematically illustrate further embodiments of the first aspect of the invention.

FIGS. 8a-8b are side cross sectional views that schematically illustrate embodiments of the second aspect of the present invention.

FIGS. 9a-9b are side cross sectional views that schematically illustrate further embodiments of the second aspect.

FIGS. 10a and 10b are graphs that illustrate a volume expansion and current response related to the second aspect.

### DETAILED DESCRIPTION OF THE PREFERRED EMBODIMENTS

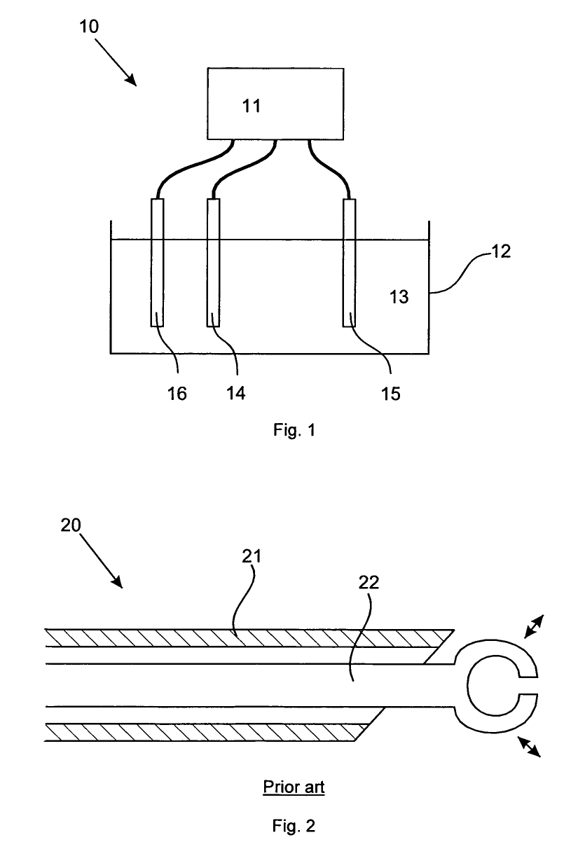

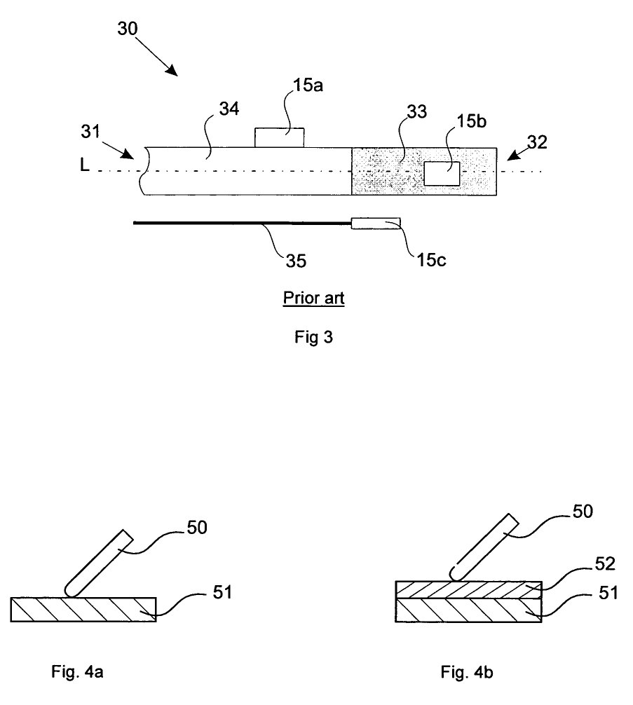

A novel class of surgical tools based on (electrical or) electrochemical actuation principles is disclosed in Published PCT Patent Application No. WO 00/78222 A1. One such tool is a micro-anastomosis connector. This is a tubular implant that is used for the reconnection of two ends of a blood vessel (micro-anastomosis). In order to activate such tools at least two electrodes are needed: a working electrode (WE) (14 in FIG. 1), which is the electroactive tool (22 in FIGS. 2 and 30 in FIG. 3), and a counter electrode (CE) 15, also known as auxiliary electrode. Conventionally, the counter electrode is formed of a metal, for instance a gold wire can be used. Also, other electrically conducting materials, coatings, or substrates can be used, such as a gold coated piece of silicon wafer. It has been shown that during surgical procedures, the tool 50 and counter electrode 51 can make accidental mechanical contact (as illustrated in FIG. 4a) that leads to electrical short circuiting, resulting in poor performance or malfunctioning of the tool.

Referring to FIG. 4b, this problem can be solved by applying an electrically insulating, ion conducting covering 52 on the counter electrode 51. Hence, ionic contact between the counter electrode 51 and the surrounding electrolyte, both the solvent and the ions, is maintained, whereas direct mechanical and electrical contact between the tool 50 and the counter electrode 51 is impossible as covering is not electrically conductive. Short circuiting is thereby prevented.

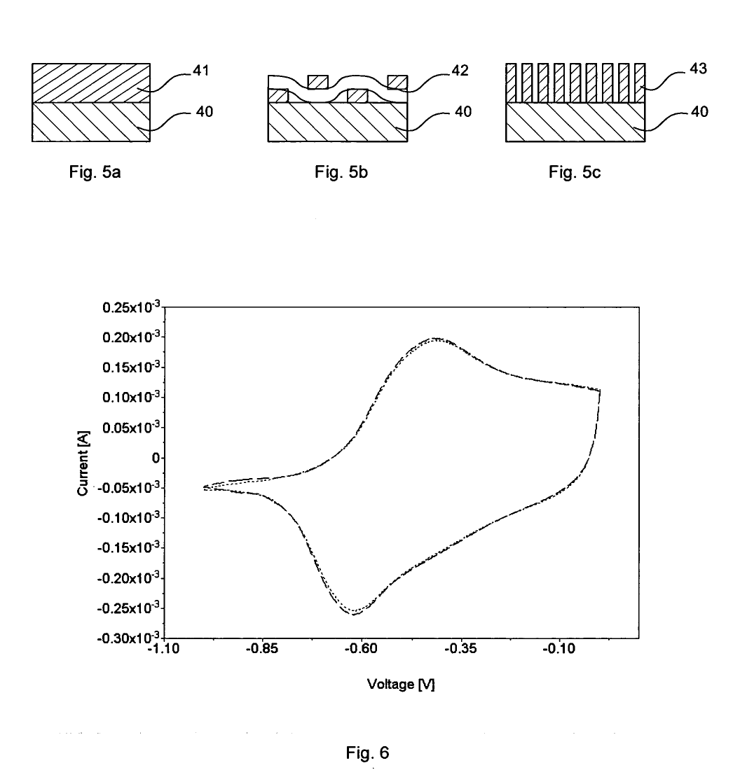

Referring to FIGS. 5a-5c, different ways of providing the covering 52 are illustrated.

In FIG. 5a, there is illustrated a covering provided in the form of a coating 41 on the electrode 40. The coating may be an ion exchange membrane, e.g. a sulfonated tetrafluorethylene copolymer, such as NAFION®, which is marketed by E.I. du Pont de Nemours and Company, Wilmington, Del., U.S.A., or FLEMION®, which is marketed by Asahi Glass Corp., Tokyo, Japan. Such materials are ion conducting and/or water conducting but electrically insulating, and may either be formed (i.e. synthesized) directly onto any standard electrode, or applied to the electrode in the form of a pre-formed film.

Hence, a standard counter electrode, such as a gold wire or a gold coated piece of silicon, may be coated, for instance using dip coating, spraying, or spin coating with an ion conducting layer. The layer may be cured in an oven at an elevated temperature according to the recipe of the material. Such coating processes are per se known to the skilled person.

In FIG. 5b, there is illustrated a covering 42 made of an insulating mesh, grid, or porous structure. Examples of such materials comprise porous materials such as Keralpor 99® from KERAFOL®—Keramische Folien GmbH, Eschenbach i.d. Opf, Germany, teflon filters, teflon mesh, etc. Yet another example of materials which may be used is Anopore®, sold by Whatman plc, Brentford, Middlesex, Great Britain.

The covering may be pre-formed and applied to the electrode, or it may be formed directly onto the electrode 40.

In FIG. 5c, the covering 43 is achieved by adding insulating structures that are patterned so that nanometer or micrometer wide channels are created that can conduct the electrolyte from the space wherein it is housed to the electrode 40. This insulating, patternable layer may be fabricated using materials such as SU8, BCB (benzocyclobutene Cyclotone®), or polyimide, either by direct photopatterning or by removing material by etching.

Such ion conducting coverings do not hinder the electrochemical reactions.

In a similar manner, the working electrode, the reference electrode, or the complete device comprising the working electrode or counter electrode, and/or any reference electrode, may be provided with the covering.

FIG. 6 shows two cyclic voltammograms of the working electrode comprising a layer of the electroactive polymer polypyrrole. A first scan (dashed line) is performed using an ordinary, uncoated gold counter electrode, and a second scan (dotted line) is performed using a Nafion coated gold counter electrode. As can be seen in FIG. 6, the graphs can hardly be told apart, and so there is no visible negative effect of the covering. The redox reaction of the PPy working electrode was exactly the same in both cases.

The inventors have recognized that it is not only in cases when the electrically activated medical device and the counter electrode are individually handled tools, separated from one and other, that the ion-conducting, electrically insulating covering of the counter electrode may be advantageous.

As mentioned, FIGS. 2 and 3 show elongate medical devices for insertion into the body, for instance such as the ones disclosed in PCT Published Application No. WO 00/78222, or US Published Application No. US2005165439. The medical device may comprises a hollow body 21, such as a catheter or cannula, through which an electrochemically activated medical tool 22 or 30 may be inserted. The counter electrode could be integrated on or in the hollow body 21, or all, or a part, of the hollow body, may be used as the counter electrode.

Alternatively, the counter electrode may be placed directly on the medical device, as illustrated in FIG. 3 at reference numerals 15a, 15b. In both cases, electrical contact (short circuiting) between the counter electrode and the electrochemically activated medical tool 22, 30 should be avoided. This can be accomplished by covering the counter electrode 15a, 15b, 15c as illustrated in for example FIGS. 5a-5c.

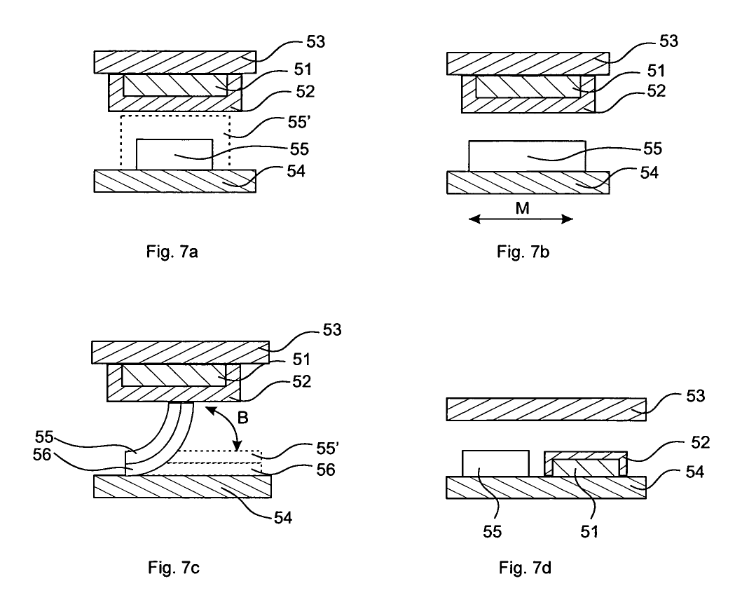

FIG. 7a schematically illustrates an other example of a device, which may be a valve device, where the counter electrode 51 may be covered to improve functionality. In this device, the volume expansion of the EAP working electrode 55, 55′ may lead to mechanical contact between the working electrode 55 and the counter electrode 51. The covering 52 prevents such electrical contact.

In FIG. 7b, yet another example is schematically illustrated. The parts 53 and 54 that comprise the counter electrode 51 and the working electrode 55, respectively, are moveable with respect to each other in direction M, for instance when parts 53 and 54 are “inter sliding” tubes, i.e. tubular devices, which interact in a telescoping manner. A covering 52 provided on the counter electrode, reduces or eliminates the risk of short circuiting.

In FIG. 7c, the mechanical contact is achieved by the movement of the EAP bi-layer actuator 55,56 (comprising the working electrode), which is fixed on a part 54. The actuator comprises two layers: an EAP layer 55 that changes volume upon activation and a non-EAP layer 56, the volume of which remains substantially constant. The volume change of the EAP layer 55, 55′ leads to a bending motion B of the actuator. If the counter electrode 51 is near or adjacent the actuator 55, 56, then the bending of the actuator may lead to mechanical contact between the actuator 55, 56 and the working electrode 51. A covering 52 provided on the counter electrode, reduces or eliminates the risk of short circuiting.

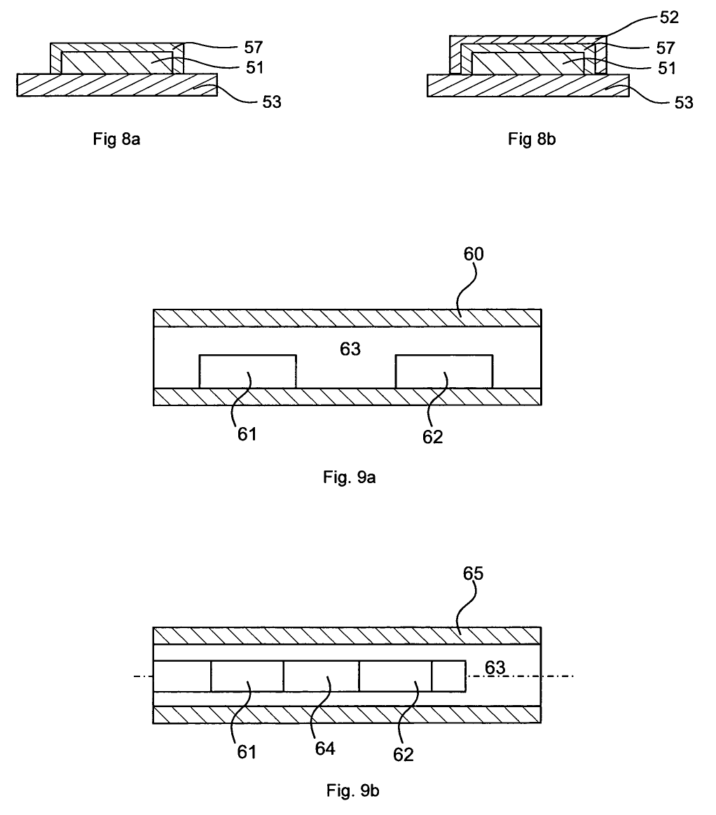

In FIG. 7d, the working electrode 55 and counter electrode 51 are both positioned on the same part 54, and electrically insulated from each other, for instance as illustrated in FIG. 3. Both electrodes may be in mechanical (and hence electrical) contact with each other through part 53. However, the ion conducting covering 52 may prevent electrical contact and thus short circuit in such instances. See also FIG. 8d, which illustrates a similar situation.

The parts 53 and 54 in FIGS. 7a-7d may be separate parts of separate devices, such as 53 being the hollow body 21 and 54 the medical device 22, 30, or two interconnected parts of a single device, such as a microfluidic channel.

As the PPy expands, it may come into mechanical and thus electrical contact with the counter electrode, leading to short circuit. If the counter electrode is coated with ion exchange layer, mechanical contact does not result in electrical contact and therewith no short circuiting occurs.

Not only the counter electrode may be covered with an electrically insulating and ion conducting coating. Likewise, the conducting polymer comprising working electrode (medical device, surgical tool) may be covered, or both the working and the counter electrode may be covered. If a reference electrode is present, this may also be covered.

In certain systems, such as can be found in catheter based applications of the polypyrrole microactuators, the total volume of the electrochemical cell may be relatively small. Activating the conducting polymer on the working electrode (actuator) may thus deplete a major part of the ions in the electrolyte available in the small electrochemical cell, especially close to the working electrode. This has several negative effects: the conductivity of the electrolyte decreases, leading to a large so called ohmic or iR drop that results in high over potentials needed for the redox process; and long diffusion paths for the ions leading to long response times and even decreased final expansion. The same negative effects occur in systems in which the ion concentration is low. In both cases, one way of supplying excess ions is by supplying a flow of electrolyte, for example by flushing the system. However, in many cases that may not be possible or desirable.

The small electrolyte volume or low electrolyte concentration situation/case can be described/defined as

V_{electrolyte}<A*VCP*CCP/C_{electrolyte}

where V_{electrolyte} is the volume of the electrolyte, C_{electrolyte} is the ion concentration of the electrolyte, VCP is the volume of the conducting polymer material, and CCP is the equivalent concentration of the ions in the conducting polymer material. A is a proportionality factor and may, for example equal 20, or 10, or 5, or 2, or 1.

The electrolyte may also be confined in a three-dimensional space, having at least one relatively small dimension.

Such small spaces may be found for example in the case where the conducting polymer operates in a tube, such as a catheter, guidewire or endoscope, of limited cross section, or in a two-dimensional space between a pair of closely spaced members, such as substantially parallel planar or curved members.

For example, at least one dimension of the space, e.g. thickness, diameter, gap distance etc., may be less than 10 times the thickness of the conducting polymer, or less than times, or less than 2 times or even less than the thickness of the conducting polymer.

In order to solve the above described problems, the counter electrode may be covered, for example by being coated, with a conducting polymer. The electrochemical system may thus have two conducting polymer electrodes, and the system can be run in a switching way: The two polymer layers are switched/activated in opposite direction. As the working electrode is oxidized, the counter electrode is reduced and vice versa. This is sometimes referred to as the rocking chair configuration.

Covering the counter electrode with a conducting polymer has several advantages. Most importantly this coating will counteract the above mentioned problem. The counter electrode will function as a second ion source/sink in addition to the electrolyte, thus reducing the ion depletion of the electrolyte near the working electrode and reducing the diffusion layer build up (ohmic or iR drop). Also, it can reduce the amount of electrolyte needed for the device, as there now exist an additional ion source/sink beyond the electrolyte. Another advantage is that the effective surface area of the counter electrode is increased, hence reducing the risk of gas formation. Also, it reduces the risk of dissolution of metal ions of the counter electrode that in turn are deposited on the working electrode. In addition, the counter electrode may be given a well defined redox reaction. Another advantage is that the conducting polymer covered counter electrode may result in lower activation potentials.

FIG. 8a illustrates an embodiment, wherein a counter electrode 51 is covered with an EAP layer 57, preferably a conducting polymer layer.

FIG. 8b illustrates yet another embodiment, where the counter electrode 51 is covered with both an EAP layer 57, preferably a conducting polymer layer, and an ion conducting covering 52, as described with reference to any of FIGS. 5a-5c.

FIG. 9a illustrates a device having a small electrolyte volume. The device 60 has a small cavity or lumen 63 that contains the electrolyte (not shown). The working electrode 61, comprising an electroactive polymer such as PPy, and the conducting polymer covered counter electrode 62 are positioned inside the device in proximity to each other. Such devices may be microchannels in microfluidics or BioMEMS applications, small tubular structures, such a catheters or cannulae, etc.

FIG. 9b illustrates a device 64 comprising the working electrode 61 and covered counter electrode 62. The device itself is inserted into or positioned inside a small cavity or lumen 63 of a separate device, object 65 or body. The device or object 65 may be a blood vessel or other body lumen, a (concentric) tube, such as a catheter, cannula, or a channel. The objects 64 and 65 may constitute (parts of) a single device. As an example, the single device may be a medical device, where reference numeral 64 designates a guide wire and reference numeral 65 designates a catheter. Alternatively, taking FIG. 2 as the example, reference numeral 64 may be the tool carrying needle 22 and reference numeral 65 may be the catheter 21 as disclosed in the PCT Published Application no. WO 00/78222.

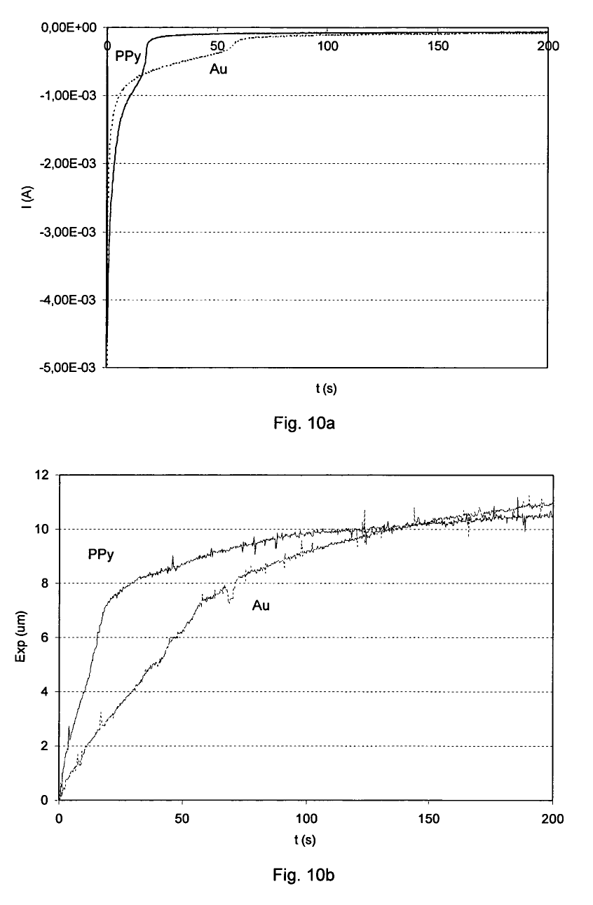

FIGS. 10a and 10b show the current and expansion response, respectively, of step activation of PPy in a small volume using different counter electrodes. At t=0 the PPy is reduced by applying −1 V. As can be seen, the response using a Au counter electrode (dashed line) is much slower (lower current and slower expansion) than using a counter electrode that has been covered with a layer of polypyrrole (solid line).

The electrolyte may be blood, blood plasma, salt solutions, contrast solutions, etc. The electrolyte may be a physiological fluid available in the area or space where the device is operated, such as blood, blood plasma, urine etc. Alternatively, the electrolyte may be an ionic solution that is externally applied to the device.

The devices described herein may be medical devices, such as catheters (such as guide catheters, balloon catheters), endoscopes, guidewires, leads (such as for cardiac rhythm management, internal defibrillators, infusion), electrodes, cannulas, embolic protection devices, introducers, sheaths, etc. The device may be a device that is temporarily inserted into the body lumen during a longer or shorter time period, or a device that is (permanently) implanted into the body.

The electroactive polymer may be a conducting polymer comprising pyrrole, aniline, thiophene, para-phenylene, vinylene, and phenylene polymers and copolymers thereof, including substituted forms of the different monomers.

Although other modifications and changes may be suggested by those skilled in the art, it is the intention of the inventors to embody within the patent warranted hereon all changes and modifications as reasonably and properly come within the scope of their contribution to the art.

1-46. (canceled)

47. An electrode system for use in a liquid electrolyte, comprising:
a first substrate member,
a second substrate member,
a working electrode arranged on one of said first and second substrate members,
a counter electrode arranged on one of said first and second substrate members,
wherein at least one of the first and second substrate members are movable relative each other and the working electrode is movable relative to the counter electrode, and
a covering at least partially provided on at least one of said working electrode and said counter electrode, said covering being ion permeable and electrically insulating.

48. The electrode system as claimed in claim 47, wherein said covering is of a material which is ion conducting and electrically insulating.

49. The electrode system as claimed in claim 48, wherein said covering is provided as a coating on said at least one of said working electrode and said counter electrode.

50. The electrode system as claimed in claim 48, wherein said covering is provided as a pre-formed part, said pre-formed part being attached to said at least one of said working electrode and said counter electrode.

51. The electrode system as claimed in claim 48, wherein said covering is formed directly on said at least one of said working electrode and said counter electrode.

52. The electrode system as claimed in claim 47, wherein said covering defines pores or channels for transporting ions.

53. The electrode system as claimed in claim 47, wherein said covering comprises a web of fibers.

54. The electrode system as claimed in claim 47, wherein said covering is removably disposed on said at least one of said working electrode and said counter electrode.

55. The electrode system as claimed in claim 47, wherein said covering is mounted relative to said at least one of said working electrode and said counter electrode so as to define a gap between said covering and said at least one of said working electrode and said counter electrode.

56. The electrode system as claimed in claim 47, wherein said working electrode is disposed on said first substrate member.

57. The electrode system as claimed in claim 47, wherein said counter electrode is disposed on said second substrate member.

58. The electrode system as claimed in claim 47, wherein said working electrode and said counter electrode are disposed on said first substrate member, and wherein said second substrate member is electrically conducting.

59. The electrode system as claimed in claims 47, wherein said first and second substrate members form separate units.

60. The electrode system as claimed in claim 47, wherein said covering is disposed on said working electrode.

61. The electrode system as claimed in claim 47, wherein said covering is disposed on said counter electrode.

62. The electrode system as claimed in claim 47, further comprising: a reference electrode.

63. The electrode system as claimed in claim 62, wherein said reference electrode is provided with said covering.

64. A method for preventing short circuiting of a working electrode, comprising the steps of:
affixing the working electrode on one of a first and a second part,
affixing a counter electrode on one of said first and second parts, said first and second parts being movable relative to one another;
covering said counter electrode in an ion permeable and electrically insulating covering; and
exposing said first and second parts to an electrolyte.

65. A method as claimed in claim 64, further comprising the steps of:
affixing a reference electrode on one of said first and second parts, said reference electrode being movable relative to at least one of said working electrode and said counter electrode, and
covering said reference electrode with an ion permeable and electrically insulating covering.

66. A counter electrode, comprising:
a counter electrode for use with a working electrode of a conducting polymer in a presence of an electrolyte, the counter electrode includes a conducting polymer.

67. The counter electrode as claimed in claim 66, wherein the conducting polymer of the counter electrode is a same material as a conducting polymer of the working electrode.

68. The counter electrode as claimed in claim 66, wherein the conducting polymer of the counter electrode is operable with a different scheme than that of the working electrode.

69. The counter electrode as claimed in claim 66, wherein the conducting polymer of the counter electrode is a different conducting polymer than that of the working electrode.

70. The counter electrode as claimed in claim 66, further comprising: a coating on the conducting polymer of the counter electrode, said coating covering at least part of the counter electrode.

71. The counter electrode as claimed in claim 66, further comprising: a covering at least partially over the counter electrode, said covering including an ion permeable and electrically insulating covering.

72. The counter electrode as claimed in claim 71, wherein the covering at least partially covers the conducting polymer.

73. A system, comprising:
a working electrode of a conducting polymer;
a counter electrode of a conducting polymer; and
an electrolyte in contact with said working electrode and said counter electrode.

74. The system as claimed in claim 73, wherein the conducting polymer of the counter electrode is same as that of the working electrode.

75. The system as claimed in claim 73, wherein the conducting polymer of the counter electrode is operable with a different scheme than that of the working electrode.

76. The system as claimed in claim 73, wherein the conducting polymer of the counter electrode is a different conducting polymer material than that of the working electrode.

77. The system as claimed in claim 73, wherein the conducting polymer is provided as a coating on the counter electrode, said coating covering at least part of said counter electrode.

78. The system as claimed in claim 73, further comprising: an ion permeable electrically insulating covering at least partially over the counter electrode.

79. The system as claimed in claim 78, wherein the ion permeable electrically insulating covering at least partially covers the conducting polymer.

80. The system as claimed in claim 73, wherein said electrolyte has a volume or ion concentration that is so small that ion depletion of said electrolyte in a vicinity of the working electrode significantly reduces system functionality.

81. The system as claimed in claim 80, wherein said electrolyte is of a volume that is less than a volume of the conducting polymer on the counter electrode, multiplied by a ratio of ion concentration of the conducting polymer to an ion concentration of the electrolyte, multiplied by a factor A, wherein the factor A is no greater than 20.

82. The system as claimed in claim 81, wherein the factor A is selected from the values of 10, 5, 2 and 1.

83. The system as claimed in claim 73, wherein said electrolyte is confined in a three dimensional space, at least one dimension of said three dimensional space being small enough to effectively limit an amount of ions available for interaction with the working electrode such that ion depletion of said electrolyte in a vicinity of the working electrode significantly reduces system functionality.

84. The system as claimed in claim 83, wherein said at least one dimension is less than 10 times a thickness of the conducting polymer of the working electrode.

85. The system as claimed in claim 83, wherein said at least one dimension is less than 5 times a thickness of the conducting polymer of the working electrode.

86. The system as claimed in claim 83, wherein said at least one dimension is less than 2 times a thickness of the conducting polymer of the working electrode.

87. The system as claimed in claim 83, wherein said at least one dimension is less than a thickness of the conducting polymer on the working electrode.

88. The system as claimed in claim 73, wherein said working electrode and said counter electrode are so closely spaced to each other that they risk contacting each other during normal operation of the system.

89. The system as claimed in claim 73, further comprising:
a first substrate member on which said working electrode is provided, and
a second substrate on which said counter electrode is provided.

90. The system as claimed in claim 89, wherein said working electrode expands upon actuation, said working electrode expanding upon actuation towards said counter electrode.

91. The system as claimed in claim 89, wherein said working electrode forms part of a bending element, said bending element being operable upon actuation to bend towards said counter electrode.

92. The system as claimed in claim 89, wherein said first and second substrate members are substantially stationary relative to each other.

93. The system as claimed in claim 89, wherein said first and second substrate members are movable relative to each other.

94. The system as claimed in claim 93, wherein said first and second substrate members interact in a telescoping sliding or intersliding manner.

95. The system as claimed in claim 73, further comprising: a tubular member to which is affixed said working electrode and said counter electrode.

96. The system as claimed in claim 95, wherein said tubular member is adapted for insertion into a body lumen.

97. A method for preventing ion depletion in an electrochemical cell, comprising the steps of:
forming a working electrode at least partially of a conducting polymer,
forming a counter electrode at least partially of a conducting polymer; and
contacting the working electrode and the counter electrode with an electrolyte.
